(19) United States
(12) Patent Application Publication
(10) Pub. No.: US 2009/0259158 A1
(43) Pub. Date: Oct. 15, 2009

(54) Antimicrobial fibers embedded with an iodinated resin

(75) Inventor: Pierre J. Messier, Quebec (CA)

Correspondence Address: GOODWIN PROCTER LLP;ATTN: PATENT ADMINISTRATOR, 620 Eighth Avenue, NEW YORK, NY, 10018, US

(73) Assignee: Triosyn Holding Inc., Williston, VT (US)

(21) Appl. No.: 12/381,328

(22) Filed: Mar. 11, 2009

Related U.S. Application Data
(63) Continuation-in-part of application No. 10/528,005, filed on Jun. 9, 2006.
(60) Provisional application No. 60/411,006, filed on Sep. 16, 2002.
(60) Provisional application No. 60/434,526, filed on Dec. 19, 2002.
(60) Provisional application No. 60/458,800, filed on Mar. 28, 2003.
(60) Provisional application No. 61/068,968, filed on Mar. 11, 2008.

(51) Int. Cl. A61L 15/46; B01D 39/08; A61L 2/235; A61L 9/00; D04H 13/00; D03D 25/00

(52) U.S. Cl. 602/48; 442/123; 442/110; 442/60; 210/501; 96/226; 604/360

(57) ABSTRACT

The present invention provides a fiber with iodinated resin particles embedded therein. The innovative fibers a show no or negligible leaching of iodine into the atmosphere and thus are not toxic. In one aspect, the antimicrobial fibers can be bonded or woven together to form a filter media. Examples of nonwoven filters are provided. Additionally, an electric charge can be applied across the nonwoven substrate to increase antimicrobial efficacy.

### CROSS-REFERENCE TO RELATED APPLICATIONS

This application is a continuation-in-part (CIP) of U.S. application Ser. No. 10/528,005, filed on Mar. 16, 2005, which is a 371 of PCT/IB03/04553, filed on Sep. 8, 2003, which claims the benefit of priority to U.S. Provisional Application Nos. 60/411,006, 60/434,526 and 60/458,800, filed on Sep. 16, 2002, Dec. 19, 2002 and Mar. 28, 2003, respectively, the contents of each are hereby incorporated by reference herein in the entirety. This application also claims the benefit of provisional application 61/068,968, filed on Mar. 11, 2008.

### FIELD OF THE INVENTION

The present invention relates to filter media with iodinated resin incorporated therein, and a method of making the same.

### BACKGROUND OF THE INVENTION

Iodine/resin demand disinfectants are known in the art. For example, U.S. Pat. No. 5,369,452 (“the '452 patent”), to Messier, the entire contents which are hereby incorporated by reference, describes a process for preparing an iodine demand disinfectant resin from an anion exchange resin. The demand disinfectant iodinated resins described in the '452 patent may be in the form of a powder (i.e. Triosyn® T-50 powder) or may be in the form of a bead (i.e. Triosyn® T-50 beads). The iodine demand disinfectant resins described in the '452 patent have a low iodine bleed characteristic since the iodine is tenaciously associated with the resin. As a result, the iodinated resins can be used in various applications as a disinfectant without the concomitant risk of leaching substantial iodide into the environment causing a high level of toxicity.

For example, antimicrobial filters can be made by incorporating an iodinated demand disinfectant in a filter media. The antimicrobial filter media can be used to retain and deactivate a large array of microorganisms. Such filter media have the advantage that the deactivated microorganisms are not released back into the environment after being used. As used in this application the term “antimicrobial filter media” includes any media that has an antimicrobial and filtering effect. Thus, these media can be used for filters per say but can also be use in making wound dressings, clothing and other useful products.

Several methods of incorporating an iodinated resin in a filter media have been described in the prior art. For instance, U.S. Pat. No. 6,224,655 discloses adhering particles of an iodinated resin to the filter media's surface with an adhesive or physically entrapping the particles in the three dimensional matrix structure of the filter media. In published U.S. patent application number 20010045398 entitled “Process For The Immobilisation Of Particles In A Three Dimensional Matrix Structure” the filter media is first produced and then an iodinated resin, is added using alcohol or a partial solvent with a high pulsation vacuum pump that opens the filter media's pores so that the iodinated resin will be physically entrapped therein.

Despite the advantages of these prior art iodinated filter media, there still exists a need to develop highly efficacious antimicrobial filter media that exhibit an even lower toxicity then the prior art iodinated filters.

### SUMMARY OF THE INVENTION

In accordance with one aspect of the present invention, there is provided a fiber with iodinated resin particles embedded therein.

In another aspect of the present invention, there is provided a filter media made from the fibers which have the iodinated resin particles embedded therein

In yet another aspect of the present invention, there is provided a filter media made from the fibers which have the iodinated resin particles embedded therein and are electrostatically charged.

In addition to the above aspects of the present invention, additional aspects, features and advantages will become better understood with regard to the following description in connection with the accompanying drawings.

### BRIEF DESCRIPTION OF THE DRAWINGS

FIG. 1 depicts aspects of an exemplary embodiment of the present invention in accordance with the teachings presented herein.

FIGS. 2 and 3 depict exemplary embodiments of electrostatically charged substrates.

FIG. 4 depicts an exemplary embodiment for providing a nonwoven media with an active agent incorporated thereon.

FIG. 5 is a two-dimensional depiction of the prior art embodiment where the iodinated resin particles are physically entrapped within the three-dimensional matrix of a nonwoven filter media.

FIG. 6 is a two-dimensional depiction of an exemplary embodiment where the iodinated resin particles are completely embedded within the fibers comprising the nonwoven.

FIG. 7 is a two-dimensional depiction of an exemplary embodiment wherein the iodinated resin particles are partially embedded within the fibers comprising the nonwoven filter media.

FIG. 8 depicts an alternative embodiment of the present invention wherein the different sized iodinated resin particulates are embedded in the fibers comprising the nonwoven filter media.

FIG. 9 is a two-dimensional depiction of a nonwoven filter media where the “kill” zone of the individual antimicrobial fibers embedded with iodinated resin is shown.

### DETAILED DESCRIPTION OF THE INVENTION

The following sections describe exemplary embodiments of the present invention. It should be apparent to those skilled in the art that the described embodiments of the present invention provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present invention as defined herein and equivalents thereto.

The present invention comprises antimicrobial fibers that are embedded with a demand disinfectant iodinated resin. It has been found that embedding an iodinated resin into a fiber imparts excellent properties to the fiber which are ideal for the production of biocidal nonwoven and woven articles. Iodinated resin powder can be extruded with various polymers to generate fibers with embedded iodinated resin. These polymers include but are not limited to polypropylene, polyethylene, PBT, nylon, polycarbonate, poly(4-methyl pentene-1), polylactic acid, and polystyrene.

It has further been found, surprisingly, that when the iodinated resin is embedded in the fiber, the amount of iodine that is released into the environment is substantially less than the amount of iodine released when the iodinated resin is in ‘free’ powder form, and hence not associated with a fiber. Moreover, when the fibers with embedded iodinated resin are incorporated into a filter media, the resultant filter leaches negligible amounts of iodide and is thus not toxic. For example, filter media made from a nonwoven material comprising fibers with embedded iodinated resin release less iodine into the environment then when the iodinated resin particles are physically adhered to the nonwoven substrate or introduced into the three-dimensional matrix of the substrate. Accordingly, the innovative nonwoven filters have much broader applicability when compared to antimicrobial nonwoven filters in the prior art.

In a particular embodiment, the filer media may be made from a fiber based material having a fibrous matrix structure; it may be a sponge like material have an open cell matrix structure; it may be flexible or inflexible; etc.

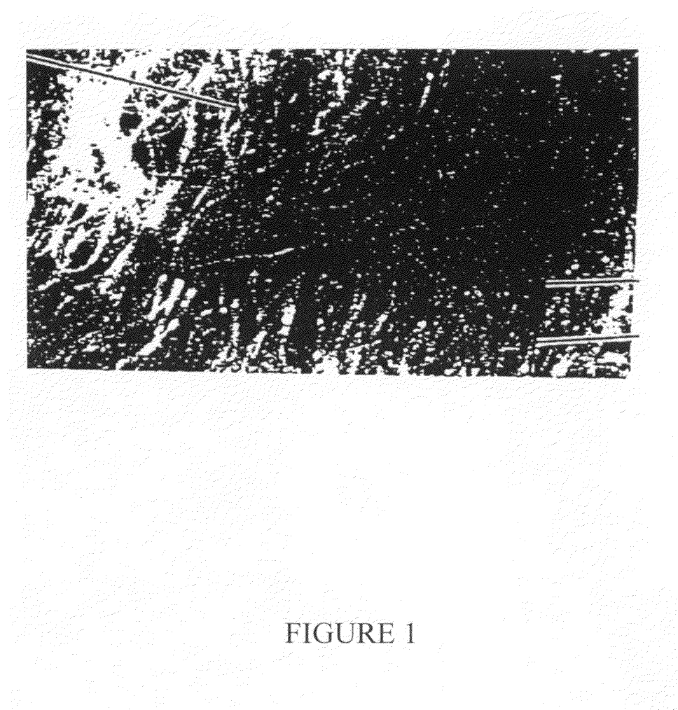

As stated above, in one embodiment, the filter media is a nonwoven fabric. Nonwoven is a type of fabric that is bonded together rather than being spun and woven into a cloth. It may be a manufactured sheet, mat, web or batt of directionally or randomly oriented fibers bonded by friction or adhesion; it may take the form of a type of fabric. FIG. 1 is provided as an exemplary embodiment of a nonwoven fabric.

In another embodiment, the filter media may be a nonwoven textile of varying fluffiness, comprising polymer fiber. The polymer may be for example, nylon, polyethylene, polypropylene, polyester, PBT, nylon, polycarbonate, poly (4-methyl pentene-1), polylactic acid, polystyrene or any other polymer suitable for a filter substrate. Additionally, the filter media can be made of materials other than polymer fiber.

The nonwoven material may be of a type suitable for a high efficiency particulate air filter (i.e. a HEPA filter). A suitable nonwoven material may be obtained from Technol Aix en Provence Cedex 03 France (see Canadian patent no. 1,243,801); another suitable material may also be obtained from Minnesota Mining & Manufacturing Co. (3M). The nonwoven material has a three-dimensional structure which should be configured in such a fashion as to provide a matrix capable of entrapping (i.e. physically) the desired active agent. For example if the nonwoven material is based on fibers, the structural fibers of the nonwoven material may be present and distributed in such a fashion as to provide a fibrous matrix structure able to entrap the desired active. In a particularly preferred embodiment, the fibers constituting the nonwoven have the active agent (e.g., iodinated resin) incorporated thereto. Accordingly, the active agent is embedded within the fibers themselves as opposed to occupying the interstitial regions formed from the bonded fibers. Alternatively, the iodinated resin particles may be embedded in the fiber and be physically entrapped within the three-dimensional matrix of the nonwoven material.

Alternative substrates may further include glass fibers and fibers, such as cellulose, that are ultimately formed into a paper-based filter media. Any substrate capable of acting as carrier for the active agent and having dielectric properties or capable of having dielectric properties imparted to it, would be a suitable substrate for the present invention. When substrates that do not have strong dielectric properties are used, such as glass fibers, additives may be provided to improve the dielectric properties of the substrate. The present invention is not limited to a nonwoven material. Other suitable substrates may include spongy materials or foam.

The active agent of the present invention may be, for example, an antimicrobial, an antitoxin, or the like. The antimicrobial may be biostatic and/or biocidal. Biostatic is a material that inhibits the growth of all or some of bacteria spores, viruses, fungi, etc. (having bioactive particles), and a biocidal is a material that kills all or some of bacteria spores, viruses, fungi, etc. Preferably, the biocidal comprises the demand disinfectant iodinated resin particles, such as those disclosed in the '452 patent. The demand disinfectant iodinated resins described may be in the form of a powder (i.e. Triosyn® T-50 powder) or may be in the form of a bead (i.e. Triosyn® T-50 beads). Owing to its ability to be incorporated into a molten mixture of polymer granules, the iodinated resin powder is particularly preferred

In a particularly advantageous embodiment, the filter media with an active agent incorporated thereto is also electrostatically charged. Accordingly, there is a potential across the surface(s) of the media creating a field wherein the field can attract and/or repel charged particles introduced to the media so that in some instances it alters the path of travel of the charged particles. Various methods can be used to incorporate an electric charge into the substrate. The charge may be induced by using a corona, needle punching, chemical enhancement, any other known charge inducing system or method, or a combination of any of the foregoing. Needle punching creates high-level friction thus adding a charge.

In a particular embodiment, to make the electrostatically charged non-woven fabric the formed media, such as felt, is placed into a corona system of about 25Kv, slow pass, until fully charged. The resulting material holds its charge for between about 6 months to 2 years.

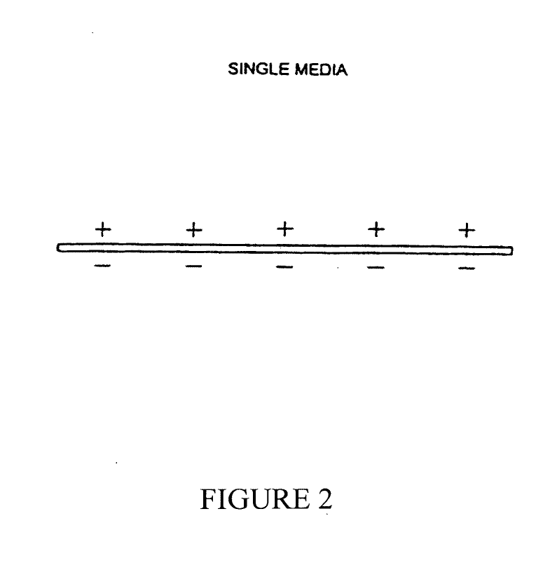

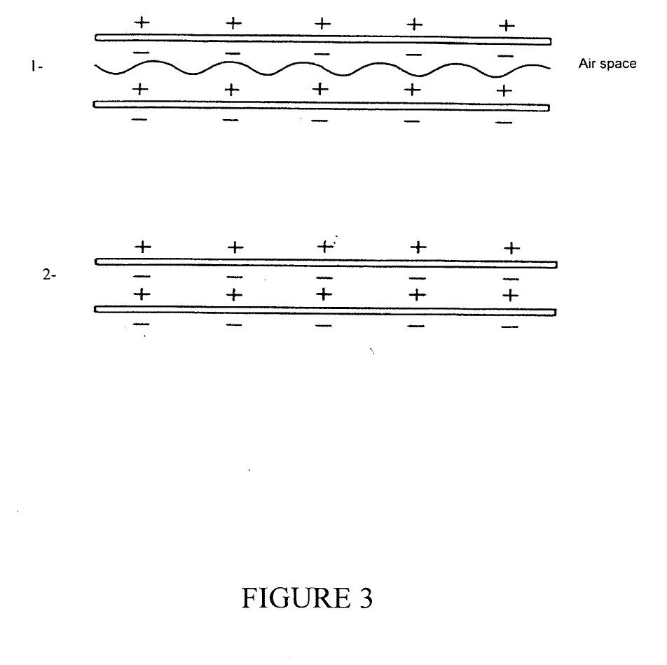

FIGS. 2-3 provide exemplary representations of electrostatically charged media. Electrostatically charged filter media of the present invention may, for example, be single or multi-layered. Each layer may be individually charged. A single layered media can have a positive charge on one side and a negative charge on the other. An example of a multi-layered media is a double-layered media. Preferably, a double layered media is used wherein the double-layered media comprises two layers, each being positively charged on one side and negatively charge on the other side, wherein the two layers are separated by an airspace and the two layers are oriented so that the negative side of one of the two layers is closest to the positive side of the other layer. In this two-layer embodiment, the air space increases the net dielectric constant of the electrostatically charged filter media.

Preferably, a high dielectric constant is provided to maintain the charge for an extended period of time. For example, air provides a good dielectric constant, as can be employed in an airspace as described above. Thus, the present invention may be effective even when wet or in a humid environment.

The resulting filter media is an insulating carrier with the active agent embedded in the fibers comprising the media. The media according to the present invention can be produced of different thickness, density and pressure drop. The media described herein can be used in, for example: clothing, wound dressings, air filters, shelters, liners and generally, any filter material.

The present invention additionally provides for a method of manufacturing the electrostatically charged filter media having an active agent incorporated thereon. The substrate itself may be manufactured according to various known methods, such as melt blown, spun blown, air laid, carted, etc. A suitable melt blown system for the above embodiment is the Accuweb provided by Accurate Products Co. of Hillside, N.J.

Prior art incorporation methods using polypropylene require the use of polyethylene to maintain a tackiness to the fibers to hold the solid particulate for a longer amount of time to prevent the particulate from falling off the fibers. In the present invention, the active agent, such as the iodinated resin disclosed in the '452 patent, are embedded in the fibers. Thus, the active agent does not have to adhere to the fibers to be incorporated into the media.

In the present invention, the active agent may be incorporated to the substrate according to various methods. For example, liquid emulsification of the active agent in the melt at increased temperature and increased pressure for mix and melt processes, or incorporation by spraying the active agent after extrusion of non-woven fibers during processing.

Various other methods of embedding the active agent in the fibers of a filter media are also suitable for the present invention. First, soaking a bail of hair-like extruded fibers in an active agent (and using alcohol to achieve the soak) and then creating the felt using pressure and temperature. Second, taking solid polymer granules manufactured with an active agent mixed in an extruder hopper to create a mixture that is then extruded into fine hair-like bails. Felt is then formed through a temperature and pressure process. Third, extruding a substrate, such as a polymer in to a hair-like substance on to which an active agent is sprayed in solid after the extrusion. The active agent may be vaporized like an aerosol. Fourth, the active agent can be injected or sprayed into non-woven fabric as the fabric is being pressurized. Fifth, carting bails of filament and mixing the resulting media with the active agent to generate a sheet having the active agent incorporated therein.

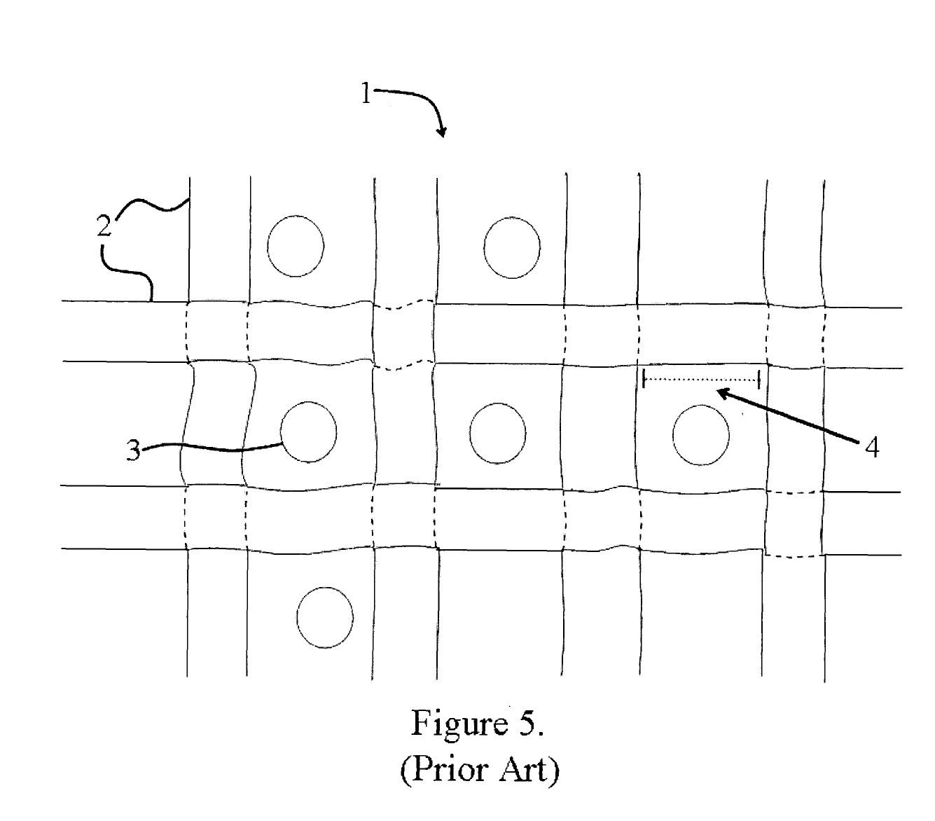

As discussed above, embedding the iodinated resin particles in the fibers of a nonwoven differs from prior art methods where resins are physically entrapped within the three-dimensional matrix of the nonwoven filter media. FIG. 5 shows a two-dimensional depiction of the prior art embodiment where the iodinated resin particles are physically entrapped within the matrix of nonwoven filter media 1. In FIG. 5, fibers 2 are bonded together to form a nonwoven. Iodinated resin particles 3 are physically entrapped within the interstitial regions 4 of the nonwoven matrix.

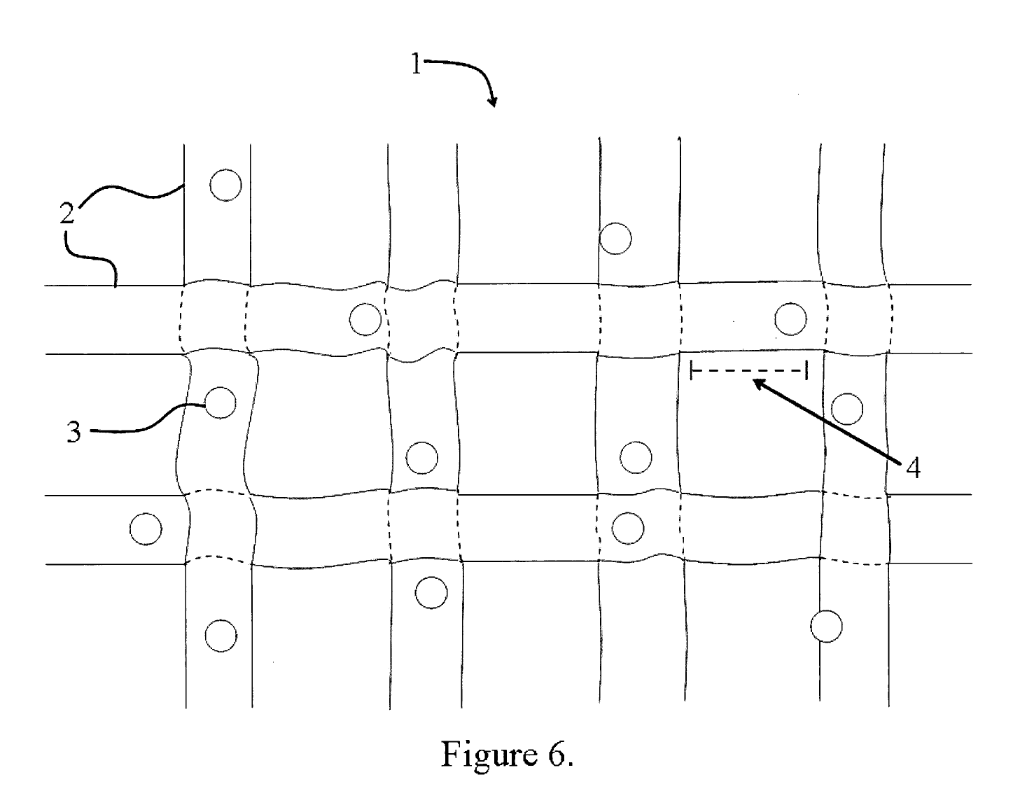

FIG. 6 is a two-dimensional depiction of an exemplary embodiment of the present invention where the iodinated resin particles 3 are completely embedded within the fibers 2 comprising nonwoven filter media 1. In this embodiment, the diameter of the fibers 2 are larger than the diameter of the iodinated resin particles 3. In this embodiment, the iodinated rein particles are not physically entrapped within the interstitial regions 4 of the nonwoven matrix

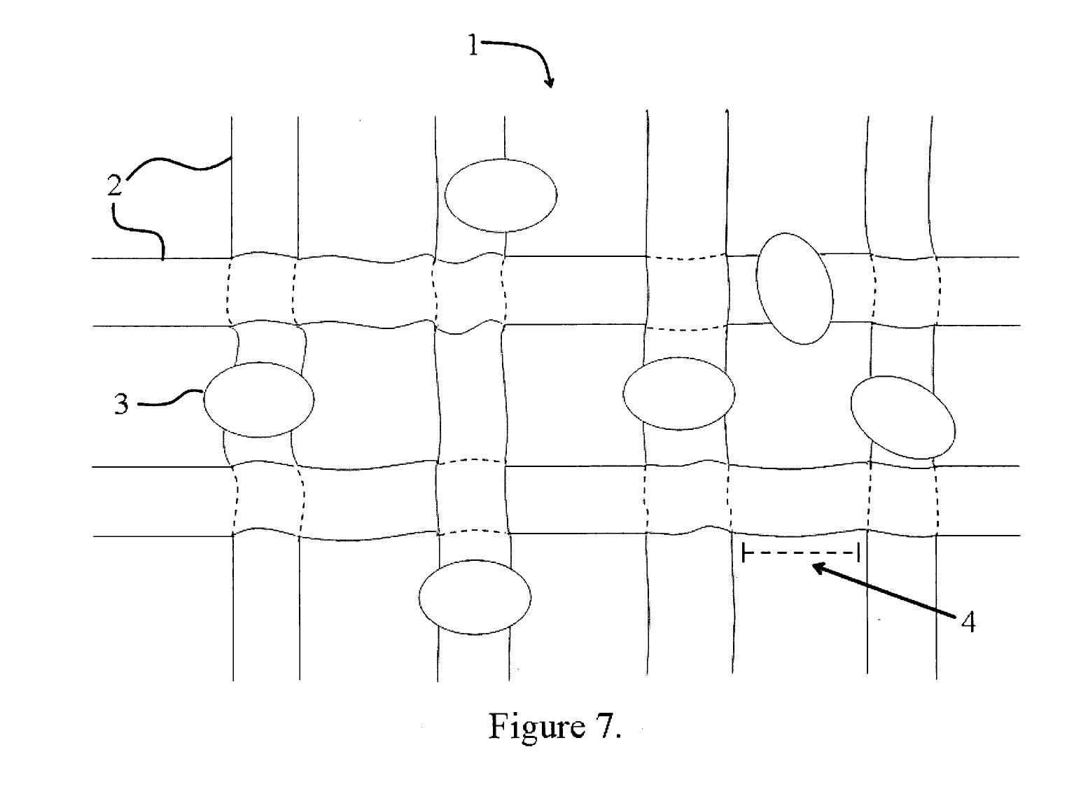

In certain embodiments according to the present invention, it may be convenient to extrude a very thin fiber with a diameter less than the diameter of the iodinated resin particulates. FIG. 7 is a two-dimensional depiction of an exemplary embodiment wherein the iodinated resin particles 3 are partially embedded within the fibers 2 comprising nonwoven filter media 1.

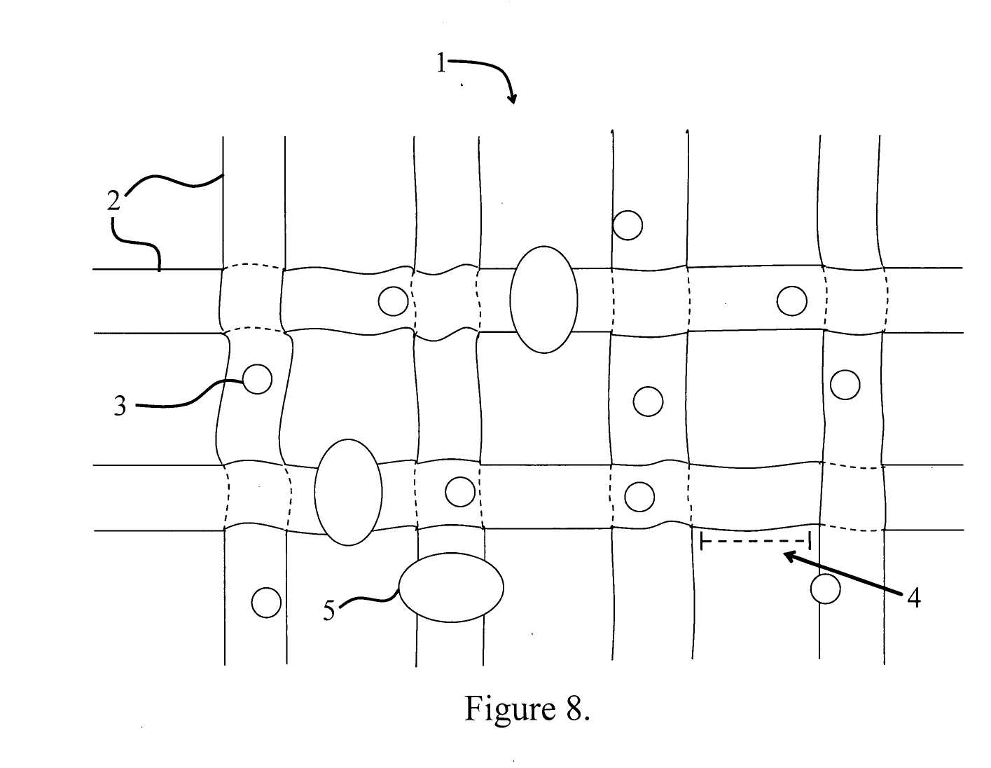

FIG. 8 depicts an alternative embodiment of the present invention wherein the different sized iodinated resin particulates 3 and 5 are embedded in the fibers 2 of filter media 1.

In another embodiment of the present invention, polymer granules are placed in a hopper of an extruder with active agent in dust form prior to extrusion. Thus, the active agent is mixed in the hopper prior to the melt. The two components are mixed, heated and then extruded to form a thin “hair” fiber used to make a felt. The resulting hair in the above embodiments having the active agent incorporated thereto is a bail-like wool. The substrate could be transparent depending on the polymer used. Additionally, a resulting polymer fiber having the active agent incorporated thereto can be treated with water, pressurized and then heated to form a felt. In other embodiments, the resulting polymer fiber having the active agent incorporated thereto can an be air laid, vacuum laid, water laid, etc.

In operation, a contaminated air or fluid stream is introduced to a filter employing the electrostatically charged filter media of the present invention. The air/fluid stream may be forced or drawn through the filter media by means of a pressure gradient. The stream may contain contaminant particles of various sizes to be removed or treated by the filter element. As the stream approaches the filter media, it is directed through the filter media such that the contaminate particles are brought into contact with the filter media and removed from the stream or treated by the active agent as describe elsewhere in this application. This is achieved through the properties of the filter, which causes the particles to follow a convoluted pathway through the filter element, thus increasing the time that the contaminant is in contact with the active agent. This increased contact time increases the effectiveness of the active agent in treating the particles in the stream.

The convoluted path that the particles follow is the result of the added electrostatic properties and the nonwoven properties of the substrate of the filter element. With respect to the electrostatic properties of the filter element, the convoluted pathway of the contaminant particles may be attributed to the particles polar nature. Polar molecules are neutrally charged and are also large in size. Because of the large size, the contaminants have a magnetic moment, which when subjected to an electric field causes the contaminant particle to be diverted from its pathway.

Additionally, the convoluted path of the contaminant particles is attributable to the nonwoven properties of the filter substrate. This is achieved because the nonwoven substrate had no direct and continuous pathway for the stream to pass through. Instead, due to the nonwoven properties, the substrate is made up of a porous material wherein no single pores of the material forms a continuous pathway through the substrate. Therefore, the stream and the particles carried by the stream are continuously diverted through the substrate.

Accordingly, the travel time through the filter is lengthened and the exposure to the active agent is increased.

The present invention can also be used in a manner consistent with existing nonwoven and woven fabrics. Uses in various goods include both durable and disposable goods. For example, nonwovens can be used products such as diapers, feminine hygiene, adult incontinence, wipes, bed linings, automotive products, face masks, air filtration, water filtration, biological fluids filtration, home furnishings and geotextiles. The media described herein can also be used in, for example: clothing, wound dressing, air filter, shelters, and liners. Additional uses include those known in the art for electrostatic filters and antimicrobial or antitoxin filters.

### Experimental Data

### A.) Iodine Release Kinetics

1.) Summary of Method

Triosyn® T-50 powder was added to a linear low density polyethylene (LLDPE) polymer and it was extruded into a fiber. A series of tests were established to compare the properties of Triosyn® (free powder) to a Triosyn®/LLDPE fiber. More specifically, the same quantity (mass) of Triosyn® T-50 powder was applied to both the LLDPE polymer as on a blank swatch of 20-XZPN to hold the Triosyn®/fiber and Triosyn powder. Note that the 50% of the mass of the total weight of the Triosyn®/LLDPE fiber is composed of Triosyn powder, (that is, 50% w/w). Both were placed into a media sandwich of blank 20-XZPN and were air filtration tested to observe the iodine release kinetics. The two swatches contained identical quantities of Triosyn® T-50 powder. The goal was to determine whether the samples would have the same iodine release profile. This quantitative test method is designed to evaluate the iodine release of test articles under an 8 hour work shift and under normal use conditions at the same flow rate.

2.) Detailed Description of Method

Prototype samples are fitted into filter holders and drilled tightly closed, attached to the sampling ports, while one sampling port is kept vacant in order to determine the iodine concentration of the challenge air stream from the chamber. Air is then passed through each sample at the desired flow rate and the quantity of iodine released is determined based on the OSHA standard protocol (Occupational Safety and Health Administration (OSHA) Iodine in Workplace Atmospheres ID#212, 1994). This test method is performed to quantify the iodine release from Triosyn® coated 12.57 cm² swatches. A High Performance Liquid Chromatograph (HPLC) equipped with an amperometry detector is used to obtain a quantifiable analysis of the iodide ions with the detection limit in the range of 0.008 to 0.0012 ppm iodide (1.3×10-3 mg/m3 to 1.9×10-3 mg/m³).

Sampling ports are connected to midget All-Glass Impingers (AGIs) containing an iodine trapping solution. A vacuum pump is employed to draw the air stream through the test articles and into the midget AGIs at a specified flow rate. The iodine released from the full devices is collected as iodide by the trapping solution contained in the midget AGIs.
At each sampling point, the impingers are removed, replaced with clean impingers containing fresh trapping solution, and the test is resumed. The recovered trapping solution is then sampled into vials for High Pressure Liquid Chromatography (HPLC) analysis.
The results from each sampling point are plotted against an iodide standard curve and the values are converted to concentration of iodine present in the sampling solution in mg/m³.
The test duration is 8 hours, which represents a common work shift period, with samplings every 15 minutes.

### Preparation of Test Articles

A sample of 0.0551 g LLDPE containing 50% w/w Triosyn® T-50 powder (10 μm) was applied onto a blank swatch of 12.57 cm2 20-XZPN. This was covered by a second swatch of the blank 20-XZPN. This was then fitted into a filter holder fabricated for such purposes, closed and placed on the rig for the iodine release kinetics testing.

A second sample was next prepared with the same swatch size, however, 0.0274 g of Triosyn® T-50 powder (10 μm) was applied directly to the blank 20-XZPN, then covered with a blank 20-XZPN. This was then fitted into a filter holder for such purposes, closed and placed on the rig for the iodine release kinetics testing. This sample will serve as the control for this test.

### Selection of Testing Flow Rate

Samples of size 12.57 cm² are air filtration tested at a flow rate of 5.4 Liters/minutes (LPM). This flow was determined based on the NIOSH (42 CFR (Code of Federal Regulations) Part 84 Approval of Respiratory Protective Devices) recommended flow rate of 85.0 Liters/minute (LPM) for the full device.

A sampling flow rate of 0.5 LPM is used as recommended by the OSHA (Occupational Safety and Health Administration (OSHA). Iodine in Workplace Atmospheres ID#212, 1994).

3.) Testing Procedure
Place the filter holders containing the media sandwiches onto the sampling ports, leaving one sampling port vacant to determine the challenge air concentration, or blank.
Add 10 mL of 1.5 mM sodium carbonate and sodium bicarbonate to each clean midget AGI-10 impinger (one per sampling port) using the Repipet® dispenser pump.
Connect the impingers to the vacuum flow meters, and the latter to the vacuum pump manifold, again using the same length of tubing.
Following a simplified air flow scheme in FIG. #1, connect the sampling ports to the impingers, using the same length of tubing for all ports. Position a Teflon membrane between the sampling port and the impinger. The Teflon membrane is approved by OSHA, to preclude any iodide containing particulates or any other types of particulates.
Calibration of the sampling flow meters should be performed before each filtration procedure. As precise and accurate flow rate readings on the aforementioned are difficult to obtain, vacuum flow meters need to be calibrated using a digital mass flow meter in order to subject the full devices to the exact selected testing flow rate.
Ensure that the digital mass flow meter is configured to measure the total volume in LPM, and that the gas is set to air.
Select a midget AGI containing the trapping solution that has been connected to its flow meter and Teflon membrane, but not to the T-connector that leads to the full device. Turn on the dilution air only and set the flow meter to 0.5 LPM. Measure the flow at the Teflon membrane using the digital mass flow meter and record the flow.
Repeat the above steps for all sampling ports to be tested, including control.
Proceed to connect the Teflon filters to the Tee-connectors.
Calibration of the total flow vacuum flow meters should be performed at the beginning of each filtration procedure and every 2 hours thereafter. As precise and accurate flow rate readings on the aforementioned are difficult to obtain, vacuum flow meters need to be calibrated using a digital mass flow meter in order to subject the test articles to the exact selected testing flow rate.
Ensure that the digital mass flow meter is configured to measure the total volume in LPM, and that the gas is set to air.
Select a port, and connect the digital mass flow meter at the end of the line where the filter holder is pulling in ambient air.
Adjust the vacuum flow meter needle valve until the mass flow meter reads the selected testing flow rate (5.4 LPM). Record the flow rate shown on the vacuum flow meter.
Repeat the above steps for all filter holders to be tested, including control ports.
At the end of the air filtration test, the sampling flow is once more recorded, and an average is taken to be used as the sampling flow in the calculations.
Set the timer to the specified interval determining the first sampling point, and the vacuum pump started. Adjust the needle valve on vacuum flow meters to the desired flow rates.
Record the temperature and relative humidity of the room.
Sample every 15 minutes for the duration of the testing period (8 hours). At each sampling point, stop the air supply to the midget AGIs. Remove the impinger bottoms for analysis, and replace with clean impinger bottoms containing fresh carbonate/bicarbonate solution.
Transfer the collected solution to a 15 mL polypropylene conical tubes, and rinse the impinger bottom with copious amounts of distilled water and then of the trapping solution. Fill with 10 mL of trapping solution using the Repipet® dispenser.
Resume the test by restarting the flow to the impinger and setting the timer to the time interval required to reach the next sampling point and continue until the test duration is completed.
Transfer 1000 μL of sample from each of the 10 mL sample solutions to an HPLC vial and then cap.
Analyze each sample by HPLC to obtain the concentration of iodide (ppm).
Calculate and record the concentration of iodine recovered from each full device from the challenge air flow using the equations presented in the next section.

### Iodine Analysis by High Pressure Liquid Chromatography (HPLC)

The method used was developed by the Dionex Corporation and validated by Triosyn Research. The HPLC is equipped with an electrochemical integrated amperometry method using an Ag/AgCl reference electrode.

The eluent is composed of 25% 250 mM sodium hydroxide solution and 75% high purity water (4 pressurized eluent bottles) previously vacuum filtered on 0.2 μm nylon filters. The high purity water is degassed prior to use and prior to preparing the basic solution.

The sample analysis is based on the injection of potassium iodide standards using a quadratic equation and the peak areas to obtain the standard curve. The standards that are regularly made are 0.001, 0.0015, 0.0020, 0.005, 0.010, 0.05, 0.100, 1.00 and 1.25 parts per million (ppm) potassium iodide in the trapping media (1.5 mM NaHCO₃/Na₂CO₃). These are made in 100 mL volumetric flasks, from a 1.0 L 100 ppm potassium iodide stock solution in high purity water. A 0.000 ppm standard must also be prepared of the trapping media alone. The standards are stable for 1 month. The samples are analyzed at ambient temperature at a flow rate of 1.5 mL/min with a run time of 5 minutes. Iodide peaks are seen at approximately 2.5 minutes.

An IonPac AS11 Guard column is used in line with an IonPac AS11 Column. The injection volume is 100 μL and the peak retention time is approximately 2.2 minutes for iodide. Columns are changed on a monthly basis. When a column is changed, it is important to start a new sequence with 3 standard curves as well as repeat injections of each standard (2 vials of each standard injected 3 times each). This is a means to monitor stability and reproducibility. Columns that are not in use are stored in 100 mM sodium hydroxide and allowed to equilibrate until needed.

The Detection limit is 1.6×10-3 mg/m³ of iodine which translates into 0.0010 ppm of iodide. The need to have such a low detection limit is driven by the low levels of iodine measured during toxicological tests. Due to the low limit, the HPLC instrument must be continuously monitored for any loss of sensitivity and kept in very good condition. These means can be met by proper maintenance and a keen sense of the life-span of the consumable parts with prompt replacement as necessary.

4.) Calculations

The HPLC instrument is a highly sensitive instrument used to determine very low concentrations of iodide species. It is the only system that could read with great precision, very small amounts of iodide species. The iodine content is calculated from the iodide measured using the following equations as per OSHA #ID-212 standard protocol.

The total amount of iodine in units of mg/m3 is determined using Equation 1 below:

$$Iodine ( mg \text{/} {m}^{3} ) = \frac{\begin{matrix}\begin{matrix}Conc . Of iodide ( mg \text{/} L ) \times \\ Trapping media vol . ( L ) \times \end{matrix} \\ Gravimetric Factor ( 1.2 )\end{matrix}}{\begin{matrix}Flow Rate ( L \text{/} min ) \times \\ \begin{matrix}Duration of Sampling ( Min . ) \times \\ {m}^{3} \text{/} 1000 L\end{matrix}\end{matrix}} \qquad EQUATION 1$$

### Where:

Conc. Of Iodide (mg/L)=Amount mg/L of Iodide from calibration curve of HPLC
Trapping media vol. (L)=Solution volume in impinger in Liters (0.0010 L in midget Impinger)
GF=Gravimetric factor=3 I2/5I−=6/5=1.2
Flow rate (LPM)=Flow of Air passing through impinger (LPM) (0.5 LPM)
Duration of Sampling (min)=Sampling time in minutes m³/1000 L=volume conversion

The total amount of iodine with units in mg comparable to the TUIL is determined from a summation of the iodine measure calculated above in mg/m³ per collection over 8 hours using Equation 2, below:

Iodine(mg)=Conc. Of iodide(mg/m3)×Breath. Rate(m3/hr)×Dur. Of sampling(min)×h/60 min EQUATION 2

### Where:

Conc. Of Iodine (mg/m³)=Calculated from previous equation
Breathing Rate (m³/hr)=1.6 m³/hr as the breathing rate during moderate activity
Duration of sampling (min)=15 minutes per sampling

Thus, over 8 hours, if collections take place every 15 minutes, a total of 32 samples of iodine in trapping solution are injected on the HPLC for iodide analysis. From the concentration of iodide, the iodine in mg/m³ is calculated (refer to equation #1) per sample, then converted into mg of iodine (refer to equation #2). The sum of all 32 samples of iodine (mg) is then taken to obtain the total amount of iodine.

5.) Results

The results obtained for the iodine release kinetics testing are presented in Table 1.

TABLE 1
IODINE RELEASED AS CONCENTRATION IN MILLIGRAMS PER CUBIC METER(mg/m³) AND
AS TOTAL IODINE RELEASED IN MILLIGRAMS (mg) OVER AN 8 HOUR AIR FILTRATION
TEST WITH SAMPLINGS EVERY 15 MINUTES AT A TOTAL FLOW OF 5.4 LPM.

| | Sample Description: | | | |
|---|---|---|---|---|
| | Sample of 0.0551 g LLDPE containing | | | |
| | 50% w/w Triosyn ® T-50 powder (10 μm) | | Sample of 0.0274 g of Triosyn ® T-50 powder (10 μm) | |
| | sandwiched between 2 swatches of | | sandwiched between 2 swatches of 12.57 cm² 20-XZPN | |
| | 12.57 cm² 20-XZPN swatches | | swatches (CONTROL SAMPLE) | |
| Time point | Concentration | | Concentration | |
| (min) | (mg/m³) | Dietary Intake (mg) | (mg/m³) | Dietary Intake (mg) |
| 15 | 0.126 | 0.050 | 2.150 | 0.860 |
| 30 | 0.090 | 0.036 | 1.788 | 0.715 |
| 45 | 0.060 | 0.024 | 1.435 | 0.574 |
| 60 | 0.051 | 0.020 | 1.209 | 0.484 |
| 75 | 0.047 | 0.019 | 1.003 | 0.401 |
| 90 | 0.042 | 0.017 | 0.828 | 0.331 |
| 105 | 0.037 | 0.015 | 0.775 | 0.310 |
| 120 | 0.037 | 0.015 | 0.768 | 0.307 |
| 135 | 0.034 | 0.014 | 0.680 | 0.272 |
| 150 | 0.032 | 0.013 | 0.654 | 0.262 |
| 165 | 0.032 | 0.013 | 0.585 | 0.234 |
| 180 | 0.027 | 0.011 | 0.581 | 0.232 |
| 195 | 0.028 | 0.011 | 0.562 | 0.225 |
| 210 | 0.028 | 0.011 | 0.552 | 0.221 |
| 225 | 0.026 | 0.010 | 0.509 | 0.204 |
| 240 | 0.024 | 0.010 | 0.487 | 0.195 |
| 255 | 0.027 | 0.011 | 0.461 | 0.185 |
| 270 | 0.024 | 0.010 | 0.460 | 0.184 |
| 285 | 0.024 | 0.010 | 0.429 | 0.172 |
| 300 | 0.024 | 0.010 | 0.437 | 0.175 |
| 315 | 0.024 | 0.010 | 0.461 | 0.185 |
| 330 | 0.023 | 0.009 | 0.460 | 0.184 |
| 345 | 0.022 | 0.009 | 0.429 | 0.172 |
| 360 | 0.029 | 0.012 | 0.375 | 0.150 |
| 375 | 0.024 | 0.010 | 0.373 | 0.149 |
| 390 | 0.025 | 0.010 | 0.359 | 0.143 |
| 405 | 0.020 | 0.008 | 0.343 | 0.137 |
| 420 | 0.020 | 0.008 | 0.338 | 0.135 |
| 435 | 0.023 | 0.009 | 0.334 | 0.133 |
| 450 | 0.022 | 0.009 | 0.324 | 0.130 |
| 465 | 0.021 | 0.009 | 0.318 | 0.127 |
| 480 | 0.021 | 0.008 | 0.314 | 0.126 |

| | | |
|---|---|---|
| Av. | 0.034 | 0.649 |
| concentration | | |
| (mg/m³) | | |
| standard | 0.022 | 0.436 |
| deviation | | |
| 8 hour dietary | 292.4 | 8167.7 |
| sum (μg) | | |

Table 1 compares the amount of iodine released over an eight hour air filtration test between the Triosyn®-embedded fiber, which is a low density polyethylene (LLDPE) polymer (50% by mass) with embedded 10 micrometer Triosyn® T-50 powder (50% by mass), compared with iodine released from free Triosyn® T-50 powder (10 micrometers). The left side of the table shows the amount of iodine released by the Triosyn®-embedded fiber while the right side of the table shows the amount of Triosyn released from the free Triosyn® powder. The results reveal that the Triosyn®-embedded fiber releases significantly less iodide than the free Triosyn® T-50 powder. The amount of iodine released is almost 30-fold lower in the Triosyn®-embedded fiber than in the Triosyn® T-50 powder.

### B.) Stagnation Leach

1.) Summary of Method

Triosyn® T-50 powder was added to a linear low density polyethylene (LLDPE) polymer and it was extruded into a fiber. The stagnation leach properties thus imparted to the Triosyn®/LLDPE fibers were then evaluated against the stagnation leach of the Triosyn® T-50 powder and Triosyn® T-50 resin beads.

2.) Detailed Description of Method

Stagnation leach testing is used as a quality control method to determine the quantity of iodine that leaches into high purity water (reverse osmosis, deionized) over a 48 hour test duration. This test is normally performed on the finished product of Triosyn® beads and is one of the control tests used to certify that the beads are acceptable for use in a range of products. In this case, the test was altered and applied to the Triosyn® powder loaded LLDPE compared to the control, Triosyn® T-50 beads and Triosyn® T-50 powder (10 μm). The results obtained will determine if absorption of the Triosyn® by the LLDPE is occurring and whether over 48 hrs, it will release the same concentration of iodine as its Triosyn® control.

3.) Testing Procedure
A quantity of 0.05 g (0.0499 g) of Triosyn® T-50 beads was weighed out on an analytical balance (±0.0003 g) into a disposable borosilicate glass test tube. The same was repeated for the Triosyn® T-50 powder (0.0501 g). 0.10 g (0.1005 g) of Triosynated (50% w/w Triosyn) LLDPE was weighed into a similar test tube.
To each test tube, 5 mL of high purity water (reverse osmosis, deionized) was added using a micro-pipette, and mixed with a glass stirring rod for 30 seconds. Each was tightly covered with parafilm at room temperature.
At 24 hours, the samples were mixed again with a glass stirring rod for 30 seconds and then allowed to settle.
At 48 hours, the samples were mixed for 30 seconds with a glass stirring rod, then allowed to settle for 30 minutes.
After the 30 minutes settling time, from each test tube 1 ml of the supernatant was removed and diluted in 4 mL of High Purity water.
DPD was added, and the absorbance of each sample was read using the spectrometer at 530 nm.
Calculations for the iodine concentration in parts per million (ppm) were performed based on equation 3 (see below) which is presented in the calculations portion of this report.

4.) Calculations

The stagnation leach standard curve was obtained using the following results presented in Table 2, and plotted on a standard curve. The absorbance for each sample is read on the spectrometer at 530 nm and 700 nm. The maximum absorbance of iodine is seen at 530 nm (top of peak) whereas 700 nm is located at the baseline. The final absorbance is obtained by subtracting A530 nm-A700 nm to obtain the true absorbance for iodine. T his is done because the baseline is not always located at zero absorbance due to shifts that may occur.

TABLE 2
ABSORBANCES AND CONCENTRATIONS MEASURED FROM
PREPARED STANDARDS OF IODINE SOLUTION.

| | | | | Iₜₒₜₐₗ |
|---|---|---|---|---|
| Sample theoretical | | | | Concentration |
| concentration (ppm) | λ530 | λ700 | λBS I₂ | (ppm) |
| 5 | 0.233 | 0.002 | 0.231 | 5.643 |
| 10 | 0.458 | 0.004 | 0.454 | 10.415 |
| 15 | 0.673 | 0.005 | 0.668 | 14.994 |
| 20 | 0.824 | 0.012 | 0.812 | 18.075 |
| 30 | 1.442 | 0.032 | 1.41 | 30.871 |

Note:
The detection limit was found to be 5 ppm.

Calculations for the concentration of iodine in the samples were performed based on the following equation (Equation 3) obtained from the above standard curve:

y=(21.398x+0.7001)×df EQUATION 3

### Where:

y=calculated concentration (ppm)
x=Absorbance measured
df=Dilution factor (in our case it was 5)

5.) Results

The results obtained from the Stagnation Leach test atr presented in Table 3. Note that each of the samples contain the equivalent amount of Triosyn®.

TABLE 3
Stagnation Leach Results Obtained From the Control Samples
of Triosyn ® T-50 Beads and Triosyn ® T-50
Powder As Well As From the Triosynated Powder

| | Triosyn ® | Triosyn ® | LLDPE/Triosyn ® |
|---|---|---|---|
| | T-50 | T-50 | Fibers (50% w/w |
| Sample I.D. | Beads | Powder | Triosyn ® |
| Results (ppm) | 5.1 | 78.9 | 4.2 |

The results of a stagnation leach test, which are displayed in Table 3, shows the same trend as the iodide release kinetics study (Table 1). That is, significantly less iodine is released INTO the water solution with the Triosyn®-embedded fiber than with the free Triosyn® T-50 powder. The stagnation leech test was also performed with Triosyn® T-50 polymer resin beads (500 micrometer). Triosyn® T-50 resin beads are produced from strongly basic ion-exchange resin beads, through methods described in the '452 patent. The amount of iodine released was comparable between the Triosyn® T-50 resin beads and the Triosyn®-embedded fibers.

The results from the iodine release kinetic study and the stagnation leach test reveal that the fiber hinders the release of iodine in a Triosyn®-embedded fiber relative to Triosyn® in free powder form. Moreover, the Triosyn®-embedded fiber is capable of holding the iodine to a similar extent to the intact Triosyn® resin beads. These results are applicable to the production of nonwoven and woven articles with antibacterial properties. Such materials must have a high degree of antimicrobial efficiency (high kill-rate) while at the same time minimizing the amount of iodine that leaches into the environment.

### C.) Antimicrobial Efficacy

1.) Summary of Method

Triosyn® T-50 powder was added to a linear low density polyethylene (LLDPE) polymer and it was extruded into a fiber. The antimicrobial efficacy was determined using the bacterial challenge, Staphylococcus aureus on 1 cm² swatches of duct tape to which extruded fibers of LLDPE containing 50% Triosyn® T-50 powder (10 μm) were applied. Triosyn® T-50 beads and the LLDPE fibers alone were also assessed. After the required incubation time, the inhibition zone represented by a clear zone in the bacterial lawn surrounding the antimicrobial-containing article was readily obtained. A zone of inhibition is a region of the agar plate where the bacteria stop growing. The more sensitive the microbes are to the test article, the larger the zone of inhibition. If the bacteria is resistant to the test article, the bacteria are expected to grow right up to the test article itself.

2.) Detailed Description of Method

The antibacterial efficacy of polymer fibers composed of LLDPE/Triosyn® was evaluated against a bacterial challenge following a method based on the disk diffusion (Kirby Bauer) protocol, developed to evaluate the susceptibility of microorganisms to antimicrobial agents. For comparison, Triosyn® beads and blank fibers composed of LLDPE alone were also evaluated. The bacterial challenge used was of Staphylococcus aureus (ATCC #6538) bacteria in the growing phase, uniformly inoculated onto a nutrient agar plate onto which the test article will be deposited, and thus exposed to the bacterial challenge.

Staphylococcus aureus is a gram positive vegetative bacterium. Its cellular morphology is roughly spherical with a mean diameter of 0.5 to 1.5 μm. S. aureus is ubiquitous in the environment and is also found in the normal flora of the human skin and nose (20-30% of the general population are carriers); it is an opportunistic pathogen primarily causing healthcare associated infections (HAIs). S. aureus is a hardy organism that withstands desiccation and can survive in dust and on certain surfaces for extended periods of time, but can be inactivated by alcohol. S. aureus is one of the reference test organisms for antimicrobial activity studies, including the standard AOAC Test Method 961.02 (Germicidal spray products as disinfectants) and ASTM Standard Test Method F2101-01 (Evaluating the Bacterial Filtration Efficiency of Medical Face Mask Materials).

Two types of polymer fibers were tested. The LLDPE/Triosyn® fibers were made from pellets composed of 50% (w/w) Triosyn® T50 powder (˜10 microns) and LLDPE, obtained through a poly-compounding process. These pellets were heated and extruded to produce fibers. The blank (negative control) fibers, composed of 100% LLDPE, were produced following the same procedure. Triosyn® T50 beads were also tested. The test articles consisted of fibers or beads placed onto the adherent side of regular duct tape (used here as a support frame) in a tightly-packed parallel arrangement to form 1.0 cm² samples.

3.) Testing Procedure

The microbiological test method used to assess the antibacterial efficacy of polymer fibers and beads was based on the disk diffusion (Kirby Bauer) protocol, developed to evaluate the susceptibility of microorganisms to antimicrobial agents:
A Tryptic Soy Broth (TSB) was inoculated with Staphylococcus aureus and incubated overnight at 35±0.5° C. under moderate agitation. 1191.
Petri dishes containing sterile Tryptic Soy Agar (TSA) were put in a laminar flow biosafety cabinet with lids ajar to remove any excess moisture.
2.0 mL of the overnight bacterial culture was put on each agar plate. The Petri dish was then rotated to ensure uniform distribution of the inoculum, then tilted to one side so that the excess inoculum could be removed using a sterile pipette.
TSA plates were allowed to dry for 15 minutes in the laminar flow biosafety cabinet, again with lids ajar.
Each test article was deposited in the middle of individual inoculated TSA plates with the polymer fibers or beads directly onto the agar surface.
TSA plates were incubated inverted at 35±0.5° C. for 18 h to 24 h.
Upon completion of incubation, the presence of an inhibition zone around the test articles was verified and the distance between the test article and the bacterial lawn (inhibition zone) were measured.

4.) Results

Inhibition Zone results are summarized in Table 4. The ability of the positive control Triosyn® polymer beads to inhibit bacterial growth was demonstrated by the 7.0 mm inhibition zone measured around the Triosyn® resin beads on the S. aureus lawn. The blank LLDPE fibers did not inhibit the growth of S. aureus and thus no zone of inhibition could be observed around the sample. In fact, growth was observed even directly below the blank LLDPE test article. In contrast, the fibers composed of Triosyn®/LLDPE were able to inhibit bacterial growth as a 1.0 mm inhibition zone was observed. Hence, the zone of inhibition using the Triosyn®-embedded fibers was approximately seven times smaller than the zone of inhibition using the Triosyn® T-50 resin beads.

TABLE 4
Inhibition Zones Around Various Polymers:
Staphylococcus Aureus Challenge (M08-0144)

| Polymers | Inhibition Zone (mm) |
|---|---|
| Blank Fibers (LLDPE) | 0.0 |
| Triosyn ®/LLDPE fibers | 1.0 |
| Triosyn ® T50 beads | 7.0 |

The results of the zone of inhibition study reveal that the Triosyn®-embedded fibers can exert a toxic effect on microbes in the region immediately surrounding the fibers. It is surprising that the zone of inhibition is significantly smaller for the Triosyn®-embedded fibers than for the Triosyn® T-50 resin beads and yet is effective in the end product, namely a woven of nonwoven fabric. This observation evidently shows that the Triosyn® resin beads are leaching more iodine than the Triosyn®-embedded fibers. Hence, the fiber serves as an effective barrier to the diffusion of iodine. Despite the fact that the iodine is held tightly to the fibers, the iodine still demonstrates a high degree of antibacterial efficiency, albeit in a highly localized region.

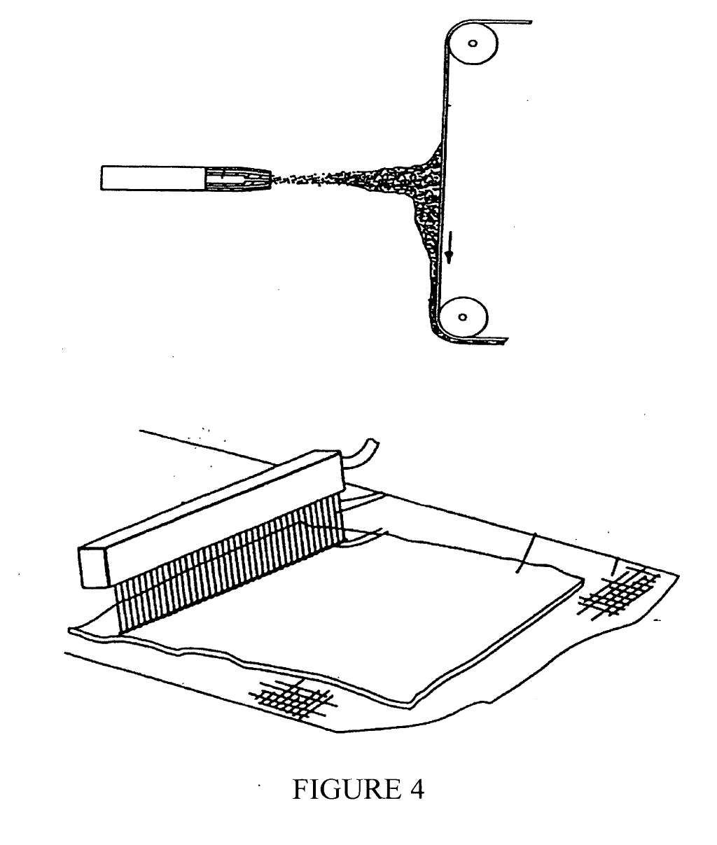

In a nonwoven article, the individual fibers, embedded with the Triosyn® powder (FIGS. 6-8), are packed together closely. A microbe passing through the nonwoven fabric thus will encounter a high concentration of iodine and will consequently be deactivated. In comparison, nonwovens that entrap iodinated resins by other methods, such as physical entrapment (FIG. 4) in the interstitial regions of a three-dimensional-matrix, would not have the same highly localized, toxic effect with minimal environmental consequences. Thus, fibers having iodinated resin embedded therein have significant advantages over other antibacterial materials such as nonwovens with entrapped iodinated resin.

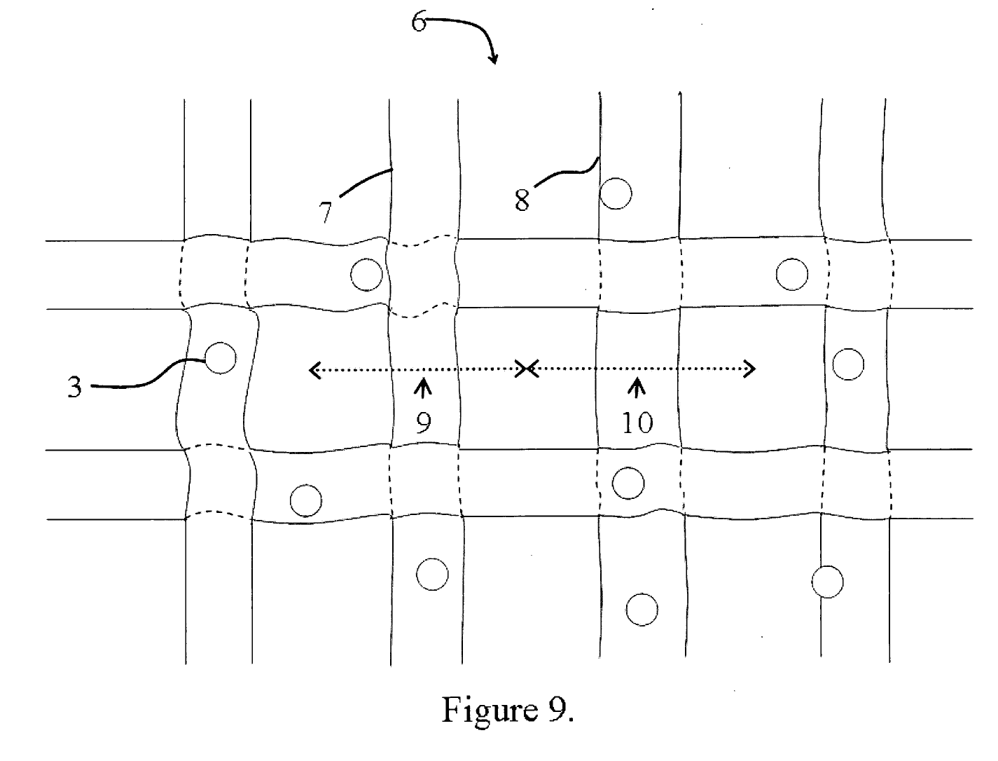

The results presented in this application have significant implications for the development of antibacterial nonwoven, woven and other fabric like materials. Because the antibacterial effect is highly localized when iodinated resin 3 is embedded in the fibers such as fibers 7 and 8 (see FIG. 9), a material 6 including fibers 7 and 8, would have a minimal degree of iodine leeching into the environment, while at the same time demonstrating a high ‘kill’ efficiency. When the fibers 7 and 8 are spaced apart from each other no more then twice the distance from the fibers 7 and 8 of zone of high “kill” efficiency, such as in a nonwoven substrate, contaminated air passing through the substrate will be disinfected in the zone of high “kill” efficiency of one or the other of the adjacent fibers 7 or 8. In FIG. 9, the zone of high kill of fiber 6 is depicted as 9 and the zone of kill of fiber 7 is depicted as 10. For instance, if the “kill” zone for each of the fibers is 2 mm, the distance between fibers ideally will be 4 mm or less. If each of the fibers 7 and 8 have different kill zones, the ideal spacing between the fibers 7 and 8 would be the sum of the two kill zones or less. A significant advantage of this invention is that smaller amounts of the iodinated resin particles may be employed in the substrate and the leach of the fiber will be reduced while still maintaining a very high level of efficacy.

Uses in various goods include both durable and disposable goods. For example, the antimicrobial fibers of the present invention can be used products such as diapers, feminine hygiene, adult incontinence, wipes, bed linings, automotive products, face masks, air filtration, water filtration, biological fluids filtration, home furnishings and geotextiles. The media described herein can also be used in, for example: clothing, wound dressing, air filter, shelters, and liners. Additional uses include those known in the art for electrostatic filters and antimicrobial or antitoxin filters.

### CONCLUSION

Having now described one or more exemplary embodiments of the invention, it should be apparent to those skilled in the art that the foregoing is illustrative only and not limiting, having been presented by way of example only. All the features disclosed in this specification (including any accompanying claims, abstract, and drawings) may be replaced by alternative features serving the same purpose, and equivalents or similar purpose, unless expressly stated otherwise. Therefore, numerous other embodiments of the additions and modifications thereof are contemplated as falling within the scope of the present invention as defined by the appended claims and equivalents thereto.

What is claimed is:

1. A filter media comprising a plurality of fibers, said fibers having an iodinated resin embedded therein.

2. The filter media as defined in claim 1 in which said media is a nonwoven material.

3. The filter media of claim 1, wherein the fibers are made from one or more polymers selected from the group consisting of polypropylene, polyethylene, PBT, nylon, polycarbonate, poly(4-methyl pentene-1), polylactic acid, and polystyrene.

4. The filter media as defined in claim 1 in which as least some of said fibers are made of polypropylene.

5. The filter media as defined in claim 1 in which the resin is in powder form.

6. The filter media of claim 2, wherein the nonwoven material is electrostatically charged.

7. The filter media as defined in claim 1, wherein the diameter of the resin is smaller than the diameter of the fibers.

8. The filter media as defined in claim 1, wherein the diameter of the resin is larger than the diameter of the fibers.

9. The filter media as defined in claim 2 further comprising particulates of iodinated resin occupying the three-dimensional interstitial regions of the nonwoven.

10. The filter media of claim 2, wherein said nonwoven material is formed into an article selected from the group consisting of a diaper, feminine hygiene product, adult incontinence product, wipes, bed lining, wound dressing and face mask.

11. The filter of claim 2, wherein said nonwoven material is a facemask.

12. The filter media as defined in claim 1 in which said media is a woven material.

13. The filter media of claim 12, wherein said woven material is an article of clothing.

14. A filter media comprising a plurality of polymer fibers having iodinated resin particles distributed therein, said fibers arranged to have an effective distance from each other in which a contamination passing said fibers is disinfected by iodine released from said iodinated resin particles, said fibers being arranged such that they are no greater than twice said effective distance from each other.

15. The filter media of claim 14, wherein the distance between fibers is no greater than the sum of the kill zones of the individual fibers.
